(19) United States
(12) Patent Application Publication
(10) Pub. No.: US 2004/0136969 A1
(43) Pub. Date: Jul. 15, 2004

(54) Adult bone marrow derived stem cells

(75) Inventor: Mehboob Hussain, New York, NY (US)

Correspondence Address: BROWDY AND NEIMARK, P.L.L.C., 624 NINTH STREET, NW, SUITE 300, WASHINGTON, DC, 20001-5303, US

(73) Assignee: New York University, New York, NY

(21) Appl. No.: 10/676,261

(22) Filed: Oct. 2, 2003

Related U.S. Application Data
(60) Provisional application No. 60/415,091, filed on Oct. 2, 2002.

(51) Int. Cl. C12N 5/08

(52) U.S. Cl. 424/93.7; 435/366

(57) ABSTRACT

The present invention relates to a method for treating a diabetic condition in a mammal by administering autologous or non-autologous bone marrow, or an effective subpopulation thereof. The present invention also relates to a method for stimulating the mobilization and differentiation of bone marrow derived cells into pancreatic islet cells.

### CROSS-REFERENCE TO RELATED APPLICATIONS

This application claims the benefit of priority from U.S. provisional application No. 60/415,091, filed Oct. 2, 2002, the entire contents of which are hereby incorporated by reference.

### BACKGROUND OF THE INVENTION

1. Field of the Invention

The present invention is directed to a subpopulation of bone marrow cells which are capable of differentiating into insulin-producing pancreatic islet cells and to a method for treating a diabetic condition by administering adult bone marrow derived stem cells which can differentiate and then function as pancreatic islet cells.

2. Description of the Related Art

Diabetes mellitus results when there is an inadequate functional mass of pancreatic beta cells. In type 1 diabetes, immune-mediated destruction of beta cells leaves a markedly reduced beta cell mass. In type 2 diabetes, there is an increased demand for secreted insulin in the face of nearly normal, but insufficient and not increased, beta cell mass. With the worldwide prevalence of diabetes increasing rapidly, there is considerable interest in finding mechanisms to increase beta cell mass by stimulating endogenous regeneration of islets (Rosenberg, et al., 1996; Rafaeloff, et al., 1995; Stoffers, et al., 2000; Guz, et al., 2001; Fernandes et al., 1997; Teitelman, 1996; Lipsett & Finegood, 2002) or by the transplantation of donor islets (Shapiro et al., 2000) or in vitro differentiated islet-like cells (Bonner-Weir et al., 2000; Halvorsen & Levine, 2001).

To this end, regeneration of pancreatic endocrine cells has been demonstrated in several experimental models (Rosenberg, et al., 1996; Rafaeloff, et al., 1995; Stoffers, et al., 2000; Guz, et al., 2001; Fernandes et al., 1997; Teitelman, 1996; Lipsett & Finegood, 2002). Multipotent stem cells have been described within pancreatic islets (Zulewski et al., 2001; Abraham et al. 2002) as well as in non-endocrine compartments of the pancreas (Bonner-Weir et al., 2000; Ramiya et al., 2000) and these cells have the capacity to differentiate into pancreatic islet-like structures in vitro. Furthermore, cells that do not reside within the pancreas, such as hepatic oval cells (Petersen et al., 1999; Yang et al., 2002) and embryonic stem cells can differentiate into pancreatic endocrine-producing cells in vitro (Lumelsky, et al., 2001) and in vivo (Soria et al., 2000) and can correct the diabetes phenotype in mice.

After transplantation of bone marrow or enriched hematopoietic stem cells, it is possible to find cells of donor origin in the skeletal, lung, gut, and skin epithelia (Krause et al., 2001), myoblasts (Ferrari et al., 1998; Gussoni et al., 1999), cardiac myoblasts (Orlic et al., 2001; Jackson, et al., 2001), endothelium (Lin et al., 2000; Asahara, et al., 1999), hepatic and biliary duct epithelium (Petersen et al., 1999; Theise et al., 2000; Lagasse et al., 2000), or neuroectoderm (Brazelton et al., 2000; Mezey et al., 2000) of recipient animals. This phenomenon of engraftment of bone marrow derived cells into multiple tissues is also found in humans (Theise, et al., 2000; Korbling et al., 2002). Thus, adult bone marrow harbors cells that have a pluripotent differentiation capacity. Recently, pluripotent mesenchymal stem cells in adult bone marrow have been described that have the capacity to differentiate into mesenchymal lineage cells, into endothelium (Jiang et al., 2002; Schwartz et al., 2002) and endoderm tissue (Reyes et al., 2001) both in vitro and in vivo (Reyes et al., 2002).

However, most studies on bone marrow derived differentiated cells are limited to morphological characteristics and gene and protein expression profiling of these cells. With the exception of cardiac and liver engraftment (Orlic et al., 2001; Reyes et al., 2001), there is little information on the functional capability of bone marrow derived cells that engraft into extramedullary tissues. Furthermore, the paradigm of bone marrow cells to be able to “transdifferentiate” into multiple cell lineages has been contested by the observation that in vitro pluripotent cells can fuse with differentiated cells and thus adapt a “differentiated” phenotype (Terada et al., 2002; Ying et al., 2002).

Several previous reports have demonstrated the presence of cells within pancreatic islets (Fernandes et al., 1997; Zulewski et al, 2001; Abraham et al., 2002), pancreatic duct tissue (Stoffers et al., 2000; Bonner-Weir et al., 2000; Ramiya, et al., 2000) and the liver (Yang et al., 2002) that have the potential todifferentiate into cells with a pancreatic endocrine phenotype. The capacity of cells, particularly cells of adult origin, to replace or supplement functional islet cells is of great interest, because of the potential role such cells could play in the treatment of diabetic conditions.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

It is an object of the present invention to provide a method for treating a diabetic condition in a mammal by administering to the mammal a therapeutically effective amount of autologous or non-autologous bone marrow, or an effective subpopulation thereof. In a preferred embodiment, the mammal is a human. The diabetic condition may be type I diabetes, type II diabetes, or a form of secondary diabetes selected from the group consisting of pancreatic diabetes, extrapancreatic/endocrine diabetes, drug-induced diabetes, lipoatropic diabetes, myotonic dystrophy-associated diabetes, diabetes induced by disturbance of insulin receptors, or diabetes secondary to one or more gene mutations or variations. In a preferred embodiment, the effective subpopulation of autologous or non-autologous bone marrow is a cellular composition consisting of greater than 20% adult bone marrow derived cells, which are depleted of hematopoietic cells and matured leucocytes and wherein such cells have a phenotype of CD45⁻, Lin⁻ and Sca⁺, as can be readily determined by RT-PCR, antibody staining and/or flow cytometry.

It is another object of the invention to provide a method for stimulating the mobilization of cells from bone marrow and the differentiation of bone marrow derived cells, preferably adult bone marrow derived cells, into pancreatic islet cells, by treating such bone marrow-derived cells with an effective stimulating amount of granulocyte colony stimulating factor (G-CSF) and/or granulocyte-macrophage colony stimulating factor (GM-CSF). This method may also be performed in conjunction with a method for treating a diabetic condition in a mammal, by administering a therapeutically effective amount of bone marrow or an effective subpopulation thereof in combination with purified recombinant G-CSF or GM-CSF in an amount effective to stimulate the mobilization and differentiation of some of the bone marrow cells into pancreatic islet cells.

It is a further object of the invention to provide an isolated subpopulation of bone marrow cells which are CD45⁻, Lin⁻, and Sca⁻ and which are capable of differentiating into insulin-producing pancreatic islet cells and a composition containing such a subpopulation.

### BRIEF DESCRIPTION OF THE DRAWINGS

FIGS. 1A and 1B illustrate the mouse bone marrow transplantation protocol. In FIG. 1A (Experiment 1), bone marrow from male INS2*EGFP mice was injected into the vascular system (or blood circulation) of irradiated female wild-type mice. In FIG. 1B (Experiment 2), bone marrow from male INS2-CRE mice was injected into irradiated female ROSA-stoplox-EGFP mice.

FIGS. 2A-2D show immunofluorescence and fluorescent in situ hybridization (FISH) results for detection of the presence of the Y chromosome in pancreatic 5-7 μm frozen sections from recipient mice of experiments 1 and 2 from FIGS. 1A and 1B above. FIGS. 2A and 2B are bright-field phase images and FIGS. 2C and 2D are composite overlay image of immunofluorescence for insulin, EGFP and FISH for Y chromosome with DAPI staining of nuclei. Y chromosome positive cells (arrows) are present in the islet (i) and in the exocrine portions of the pancreas (e). EGFP is present only in insulin positive cells in the islet. The scale in FIG. 2A corresponds to 10 μm at 400× magnification. Arrows indicate cells containing a Y chromosome. Inset in FIGS. 2C and 2D are magnified areas of EGFP and insulin double positive cells containing a Y chromosome.

FIGS. 3A-3C show immunofluorescence and FISH of isolated, dispersed pancreatic islet cells from experiment 1 (See FIG. 1A). Images of identical respective fields in subpanels A: bright field phase; subpanel B: EGFP (note slight autofluorescence of isolated islet cells); subpanel C: immunostaining with rhodamine X labeled secondary antibody for FIG. 3A: insulin; FIG. 3B: IPF-1; FIG. 3C: HNF3β; subpanel D: FISH for Y chromosome and nucleus stain with DAPI. Presence of Y chromosome is only found in cells positive for EGFP. The scale in subpanels A corresponds to 5 μm at 630× magnification.

FIGS. 4A-4C illustrate the results of cell sorting and fluorescence analysis. FIG. 4A: peripheral blood nucleated cells (PBNC); FIG. 4B: bone marrow cells; FIG. 4C: isolated and dispersed islet cells. Subpanels depict respectively: A. representational scatter-plot of side (X-axis) forward (Y-axis) scatter of respective cells. Subpanels B-D depict fluorescence scans (X-axis EGFP, Y-axis: phycoerythrin (red) filter) of respective cells. Subpanel B shows cells from INS*EGFP donor mice (experiment 1). Subpanel C shows cells from irradiated WT mice transplanted with bone marrow from INS*EGFP mice (experiment 1). Subpanel D shows cells from irradiated ROSA-stoplox-EGFP transplanted with bone marrow from INS2-CRE mice (experiment 2). No fluorescence is detectable in donor or recipient PBNC or bone marrow cells. EGFP is detectable in islets of donor animals of experiment 1 and in the recipients of experiment 1. In subpanels B-D cells in the line equidistant from both axes show some autofluorescence and cells in the lower right quadrant have EGFP signal. See FIGS. 1A and 1B for description of experiments 1 and 2.

FIGS. 5A-5C displays results of RT-PCR analysis of isolated and FAC-sorted cells from experiment 1. In FIG. 5A, the peripheral blood nucleated cells (PBNC) of recipient mice express cyclophilin (lane 1) as well as CD45 (lane 2). EGFP-expressing cells derived from pancreatic islets express cyclophilin (lane 3), lack CD45 (lane 4), and express insulin (lane 5). In FIG. 5B, EGFP-expressing cells derived from pancreatic islets express cyclophilin (lane 1), insulin I (lane 2), IPF-1 (lane 4), and HNF3β.(lane 5). Lane 3 is empty. In FIG. 5C, EGFP-expressing cells derived from pancreatic islets express cyclophilin (lane 1), insulin II (lane 2), insulin I (lane 3), GLUT 2 (lane 4), HNF 1α(lane 5), HNFlβ(lane 6), and pax6 (lane 7).

FIG. 6 shows measurement of insulin secretion after glucose or after glucose and exendin-4 stimulation of isolated EGFP positive cells from experiment 1 (dark bars). Control cells were isolated from an INS*EGFP mouse and treated identically (light bars). One thousand dispersed islet cells were collected for EGFP expression by fluorescence activated cell sorting, or manual selection of EGFP expressing cells under a fluorescence microscope, cultured for 24 hours at 5.5 mM glucose before further incubation in 5.5 mM or 11.1 mM glucose or for 10 hours. Additional cells were incubated in 11.1 mM glucose for 10 hours before exposure to 10 nM exendin-4 during the four last hours. Supernatant was collected for assay of insulin (ELISA). Results of three separate assays performed in duplicate (i.e., two samples of each experiment were measured side by side to eliminate intra-assay errors) are shown as mean±SD.

FIGS. 7A-7D show calcium signaling in EGFP-expressing cells from isolated islet cells. Single EGFP-expressing cells isolated from pancreatic islets were obtained from mice that received bone marrow from INS2*EGFP donors. FIG. 7A: spontaneous fluctuations of intracellular calcium concentration [Ca²⁺]ᵢ at ambient 11.1 mM glucose. FIG. 7B: reduced frequency and amplitude of [Ca²⁺]ᵢ fluctuations with no added glucose. FIG. 7C: an increase in [Ca²⁺]ᵢ was observed upon stimulation with extracellular glyburide (200 nM for 90 s). FIG. 7D: extracellular application of KCl (56 mM for 30 s) produced an increase in [Ca²⁺]ᵢ. In FIGS. 7A and 7B, the measurements are obtained from the same cell.

### DETAILED DESCRIPTION OF THE INVENTION

Adult bone marrow-derived cells appear to have the capacity of differentiating directly into insulin-producing cells and may replace pancreatic islet cells. Alternatively, these cells may also enter an intermediary pool of one (Guz, et al., 2002) or more multipotent cell phenotype(s) within the endocrine or non-endocrine compartments of the pancreas or as oval cells within the liver or pancreas (Yang, et al., 2002; Reddy et al., 1984; Rao & Reddy, 1995; Dabeva et al., 1997). Then, in a further step these cells may respond to local (Bonner-Weir et al., 2000; Abraham, et al., 2002; Czyz & Wobus, 2001) or circulating signals (Flier et al., 2001) and differentiate into insulin-producing cells.

Adult bone marrow harbors cells that have pluripotent differentiation capacity. Such cells, when transplanted, have the potential to restore function of certain endocrine cells to a patient who has lost such production due to disease such as diabetes mellitus. The present application contains the demonstration that bone marrow derived cells can be seen to populate pancreatic islets of Langerhans. When purified from islets, said cells express insulin, the glucose transporter 2 (GLUT2), and transcription factors typically found in pancreatic beta cells.

Furthermore, under in vitro conditions, these bone marrow derived cells exhibit glucose-dependent and incretin-enhanced insulin secretion and respond to extracellular glucose, KCl, and sulfonylurea with an increase in the concentration of intracellular calcium, just as do pancreatic islet beta cells. These results establish that bone marrow harbors cells that can differentiate into functionally competent pancreatic endocrine beta cells and thus represent a source for cell-based treatment for diabetes mellitus. Additionally, these cells provide a potentially unlimited source of islet cells without the problems of tissue rejection. Apart from the therapeutic implication of these findings, the model described here might also allow examination of the mechanisms underlying the homing of cells as well as factors controlling the regulation of gene expression in bone marrow derived cells which enter the extramedullary environment.

The present invention provides a method of treatment for a diabetic condition in a mammalian patient requiring a pancreatic islet cell transplant in an amount sufficient to reconstitute the patient's functional beta cells. The patients in need of this product are those with a specific requirement for pancreatic islet beta cells. For example, patients with Type 2 diabetes mellitus or any other diabetic condition may benefit therefrom.

In general, the present invention will comprise treating and/or preventing a diabetic condition in a mammal, e.g., a human, having or at risk of developing the diabetic condition, by administering to the mammal a therapeutically effective amount of autologous or non-autologous bone marrow, or an effective subpopulation thereof.

The present invention further provides a method for treating and/or preventing a diabetic condition in a mammal in need thereof by administering to the mammal a therapeutically effective amount of autologous or non-autologous bone marrow or an effective subpopulation thereof, wherein the autologous or non-autologous bone marrow, or effective subpopulation thereof, is administered with purified recombinant G-CSF and/or GM-CSF in an amount effective to stimulate the mobilization and differentiation of some of the bone marrow cells into pancreatic islet cells.

While autologous bone marrow, or an effective subpopulation thereof is preferred, it should be understood that in certain circumstances it may be possible to use non-autologous bone marrow populations. Most preferable would, of course, be syngeneic bone marrow, when circumstances permit. Other allogeneic bone marrow that will not be rejected by the recipient is also usable, particularly, so-called “type-matched” bone marrow that has been subjected to HLA tissue typing. If necessary, when using non-autologous bone marrow, steps may be taken to diminish the possibility of rejection, as is well known in the art.

Although the use of xenogeneic transplants is still very much a nascent art, long-term multi-lineage bone marrow chimerism has been demonstrated in a mouse to rat bone marrow transplant model (Mohiuddin et al., 2001) and similar work has been reported in primates (e.g. Allen et al., 1997). As a striking indicium of the strength of the interest the clinical community has in such transplants, Phase I clinical trials were initiated for the use of baboon bone marrow for human HIV patients (e.g. Lein, 1996).

The effective sub-population that may be administered is any sub-population of bone marrow which is enriched in the pluripotent cells which home to the pancreas and differentiate into insulin-producing β-cells. Thus, such a sub-population may be one depleted of mature cells, such as mature leukocytes, or one depleted of hematopoietic cells, or depleted of both. Sub-populations which are enriched in cells with markers denoting stem cells or free of markers denoting differentiated cells may also be used. For example, the CD45⁺ marker is indicative of differentiated cells. Thus, a CD45⁻ population would be expected to be enriched in the cells of interest capable of differentiating into insulin-producing cells. Furthermore, the effective subpopulation used in the method of the present invention are those which are not only CD45⁻ but are also Lin⁻ (lineage minus phenotype that lack three different differentiation markers) and Sca⁺ (positive for stem cell antigen surface marker associated with pluripotency). Such an effective subpopulation is considerably less than 5% of the population of bone marrow cells.

Another aspect of the present invention relates to an isolated subpopulation of bone marrow cells which is CD45⁻, Lin⁻, and Sca⁺ and which is capable of differentiating into insulin-producing pancreatic islet cells. A further aspect of the present invention provides a composition containing this isolated subpopulation of bone marrow cells and a pharmaceutically-acceptable carrier, excipient, diluent or auxiliary agent.

The enriched cell sub-population can contain as little as 20% of cells of the desired phenotype (CD45⁻, Lin and Sca⁺) or greater than 30%, 40%, 50%, 60%, 70%, 80%, 90% or 95% of cells of the desired phenotype.

Additional methods for determining the effective subpopulation include such protocols familiar to one of ordinary skill in the art as negative selection, in which antibodies for cell surface markers stain a population of lineage-committed cells according to their different specificities. Selection is then performed to enrich for uncommitted hematopoietic pro-genitors, with the expectation that such cell populations will be most likely to contain cells with the potential to differentiate into pancreatic endocrine cells. Another way the skilled artisan might pinpoint such cells of interest would be by examination of a so-called “side population” of cells seen in flow cytometric analysis. Such populations often define potent hematopoietic stem cells (Jackson et al., 2001).

Yet another way by which the effective subpopulation of the present invention might be characterized is by selecting cell subpopulations in culture. It is understood that bone marrow stem cells either display a self-renewing phenotype, which maintains stem cell properties, or, produce cells that will differentiate further into the different hematopoietic cell fates (Krause, 2002). The former subpopulation is pluripotent and may be of special interest to the artisan in finding insulin-producing cells.

The present invention will now be further illustrated by reference to the following examples which are provided solely for purposes of illustration and are not intended to be limiting.

### EXAMPLES

Bone marrow harbors cells that have the capacity to differentiate into cells of nonhematopoietic tissues of neuronal, endothelial, epithelial and muscular phenotype. In this example, the laboratory of the present inventor demonstrates that bone marrow derived cells populate pancreatic islets of Langerhans. Bone marrow cells from male mice that express—ultilizing a CRE-LoxP system—an enhanced green fluorescent protein (EGFP) if the insulin gene is actively transcribed were transplanted into female, lethally irradiated recipient mice. After 4-6 weeks post transplantation, recipient mice revealed Y-chromosome and EGFP double-positive cells in their pancreatic islets. Neither bone marrow cells nor circulating peripheral blood nucleated cells of donor or recipient mice had any detectable EGFP. EGFP-positive cells purified from islets express insulin, the glucose transporter 2 (GLUT2) and transcription factors typically found in pancreatic β-cells. Furthermore, in vitro these bone marrow derived cells exhibit—as do pancreatic β-cells—glucose-dependent and incretin-enhanced insulin secretion and respond to extracellular glucose, KCl, and to a sulfonylurea with an increase of intracellular calcium. These results demonstrate that bone marrow harbors cells that have the capacity to differentiate into functionally competent pancreatic endocrine β-cells and represent a source for cell-based treatment for diabetes mellitus. The CRE-LoxP system (Saver et al., 1998) was used in the present study to allow identification, purification and functional characterization of insulin-expressing cells from pancreatic islets that have been derived in vivo from transplanted bone marrow. These cells express insulin, the glucose transporter 2 (GLUT2), and transcription factors typically found in pancreatic βcells. These bone marrow derived cells have electrophysiological properties typical for pancreatic β-cells and secrete insulin in response to a glucose stimulus or to the incretin hormone analog exendin-4. The results generated with the Cre-LoxP system also contest the notion that cell fusion is in vivo an explanation for the “transdifferentiation” of bone marrow derived cells into differentiated cell phenotypes.

The following materials and methods are applicable to all of the following examples.

Materials and Methods

Murine Model of Bone Marrow Transplantation and Experimental Design

Animals were maintained under conditions approved by the institutional Animal Care and Use Committee at New York University School of Medicine. Transgenic mice expressing the phage CRE recombinase (CRE) under control of the rat insulin 2 promoter (INS2-CRE) (Gannon et al., 2000) were obtained from Jackson Laboratories (Bar Harbor Me.; C57Bl/6 background). Mice expressing EGFP at the ROSA26 locus (Soriano, 1999) preceded by three floxed translation stop codons (ROSA-stoplox-EGFP; C57Bl/6 background) (Mao, et al., 2001) were obtained from G. Eberl and D. Littman, New York University School of Medicine. The stop codons are removed by activity of the CRE that recognizes the LoxP sequence, removes the chromosomal portion flanked by the LoxP sites (i.e. translation stop sequences), thus resulting in a permanent expression of EGFP (Sauer & Henderson, 1988). To generate INS2*EGFP mice, heterozygous INS2-CRE mice were crossed with hemizygous ROSA-stoplox-EGFP mice and offspring were genotyped at time of weaning. In INS2*EGFP mice, cells that activate the insulin promoter express CRE and become identifiable by their expression of EGFP. Genotyping was performed with following primers according to standard protocols:

```
EGFP (fw: 5′-gcgagggcgatgccacctacggca-3′ (SEQ ID NO:1);
            rv: 5′-gggtgttctgctggtagtggtcgg-3′ (SEQ ID NO:2); 450 bp),
CRE (fw: 5′-taaagatatctcacgtactgacgg-3′ (SEQ ID NO:3);
            rv: 5′-tctctgaccagagtcatccttagc-3′ (SEQ ID NO:4); 250 bp).
```

Bone marrow from male donor mice obtained from the medullary cavities of tibias and femurs using a 25G needle was flushed under sterile conditions with Hank's Balanced Salt Solution (HBSS). Marrow cells were checked for viability with trypan blue and elutriated as previously described(Krause et al., 2001). Recipients were irradiated female mice, exposed to 1050 to 1100 cGy (from a gamma cell 40 small animal irradiator, Atomic Energy of Canada, Kanata, Ontario, Canada). They received approximately 104 unfractionated bone marrow derived cells via tail vein injection. Recipient mice were kept in sterile cages with sterile chow and water for 2 weeks postirradiation and were euthanized at 4-6 weeks after bone marrow transplantation for tissue harvesting. Because all animals had the same C57BL/6 background, no alloimmune or graft-versus host response was expected (or observed). Engraftment was checked by determining the fraction of peripheral blood nucleated cells positive for a Y chromosome (2 counts of 100 nucleated cells per animal).

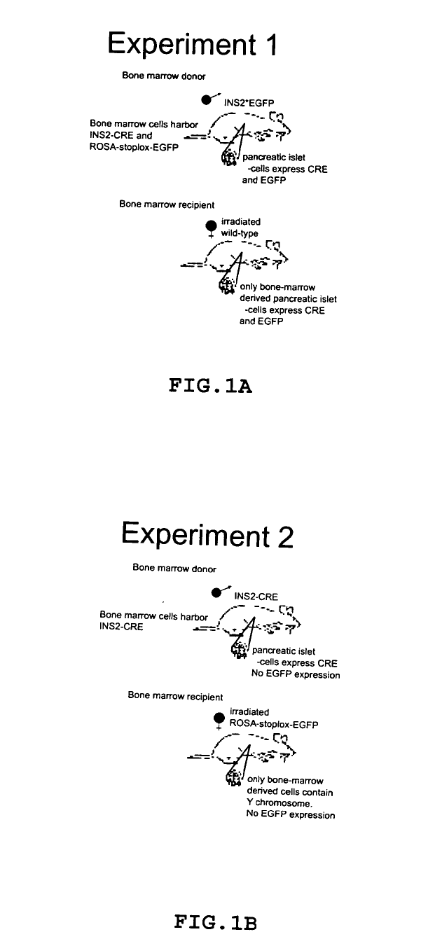

Experimental bone marrow transplantation procedures are summarized in FIGS. 1A and 1B. In one set of experiments, donor animals were male INS2*EGFP mice, and recipients were female wild-type (WT) mice (FIG. 1A; experiment 1). In a second set of experiments, donor animals were male INS2-CRE mice, and recipients were female ROSA-EGFP mice (FIG. 1B; experiment 2). Experiment 2 tests for EGFP expression in recipient cells secondary to any cell fusion with donor cells expressing CRE.

Islet Cell Iolation

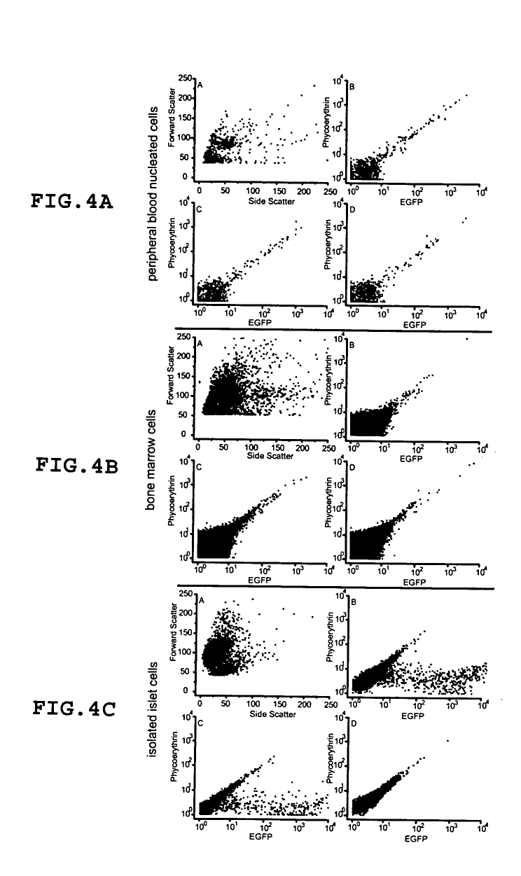

Islets were isolated using the collagenase method as previously described (Hussain & Habener, 2000). Briefly, the pancreas was injected with 1 cc of collagenase P (2 mg/ml) (Roche Diagnostics, Indianapolis, Ind.) and DnaseI (0.5 mg/ml) (Roche Diagnostics). The pancreas was then digested at 37° C. in a total of 10 ml of digestion solution under constant shaking and intermittent vortexing. Subsequently, islets were washed several times in HBSS with bovine serum albumin (5 mg/ml). Islets were hand picked under a dissecting microscope. Islets were dispersed into single cells by suspension in trypsin EDTA (GIBCO BRL, Grand Island N.Y.) and triturated through a siliconized Pasteur pipette. Cells were then cultured in RPMI 1640 (Gibco-BRL, Grand Island, N.Y.) with 5.5 mM glucose and 10% fetal calf serum added in a humidified incubator (95% air, 5% CO₂) at 37° C. EGFP-positive islet cells were isolated from EGFP-negative cells by fluorescent activated cell sorting (FACS) (Becton-Dickinson, Franklin Lakes, N.J.) for further culture, RT-PCR (see below) or immunostaining and fluorescent in-situ hybridization (FISH). FACS data are given in FIG. 4 and were generated on the WinMDI 2.8 software (Scripps Institute FACS core facility server, La Jolla, Calif.). Units of X- and Y-axes are arbitrary but were kept the same for all experiments. Subpanel A of FIG. 4 has linear axes and shows forward- and side scatter of cells. Subpanels B-D have X and Y-axes in logarithmic scale show green fluorescence intensity in the X-axis (EGFP signal) and phycoerythrin (red) fluorescence intensity in the Y axis (phycoerythrin signal).

Immunohistochemistry and Fluorescent In-Situ Hybridization (FISH)

Tissues were fixed in 4% paraformaldehyde for 2 hours at room temperature and subsequently in 30% sucrose overnight at 4° C. before embedding in Tissue-Tek optimal cutting temperature (OCT) compound (Sikura, Torrance, Calif.) for sectioning into 5-7 μm sections. A total of 200 pancreatic sections were analyzed. Isolated islet cells were fixed on microscope slides with 4% paraformaldehyde for four minutes at room temperature. Tissue sections and cells were treated with 100% methanol for 2 min at −20° C. and then blocked with 3% normal donkey serum for 10 min at room temperature. Samples were then incubated at 4° C. overnight with non-immune serum, guinea pig antiserum raised against insulin (Linco Research St. Louis, Mo.) at a dilution of 1:1000, rabbit anti-IPF-1 (1:500) (Hussain & Habener, 2000), or rabbit-anti HNF-3β.(1:2000). After rinsing with PBS, slides were blocked again with 3% donkey serum for 10 min at room temperature before being incubated with donkey anti-guinea pig or donkey anti-rabbit with conjugated rhodamine X (Jackson Immunoresearch Laboratories, West Grove, Calif.)(1:500) for 30 minutes at room temperature. Slides were mounted in fluorescent mounting medium (Vector Laboratories, Burlingame, Calif.) and stored in the dark. For frozen sections, a rabbit anti-GFP IgG conjugated with Alexa Fluor 488 (Molecular Probes, Eugene Oreg.) at a dilution of 1:2000 was used to detect presence of EGFP-positive cells. After image capturing, the same samples were analyzed for the presence of mouse Y chromosome with FISH with Rainbow*FISH (Cambio, Cambridge, UK) according to the manufacturer's recommendation. A digestion with proteinase K (10 μg/ml at 37° C., 5 minutes) (Sigma-Aldrich, St. Louis, Mo.) was added at the beginning of the FISH protocol. Nuclei were stained with 4′,6-diaminidino-2-phenylindole (DAPI) using fluorescent mounting medium containing DAPI 1.5 μg/ml (Vector Laboratories, Burlingame, Calif.).

Imaging was performed on a Zeiss Axioskop 2 fluorescent microscope (Carl Zeiss MicroImaging, Thornwood, N.Y.) equipped with a cooled CCD digital camera (Hamamatsu Orca; Hamamatsu Photonics K K, Hamamatsu City, Japan) and Improvision Open Lab software (Improvision Scientific Imaging, Lexington, Mass.) that allows pseudocoloring. Images were captured using the appropriate light absorption and emission filters supplied by the manufacturer of the microscope.

RT-PCR Analysis

A subset of fluorescence sorted islet cells were taken in culture for 24 hours and then hand picked twice for positive EGFP fluorescence. One hundred of these cells were used for RT-PCR analysis. Total RNA from cells was purified using RNeasy with an Rnasefree Dnase digestion step (Qiagen, Valencia, Calif.). Reverse transcription was performed with Omniscript (Qiagen), and PCR was performed using recombinant DNA polymerase (TaKaRa Taq, Shiga, Japan). Primers and PCR product size are as follows:

```
insulin I (fw: 5′-tagtgaccagctataatcagag-3′ (SEQ ID NO:5);
                                rv: 5′-acgccaggtctgaaggtcc-3′ (SEQ ID NO:6); 288 bp),
insulin II  (fw: 5′-ccctgctggccctgctctt-3′ (SEQ ID NO:7);
                                rv: 5′-aggtctgaaggtcacctgct-3′ (SEQ ID NO:8); 212 bp
IPF-1 (fw: 5′-tgtaggcagtacgggtcctc-3′ (SEQ ID NO:9);
                                rv: 5′-ccaccccagtttacaagctc-3′ (SEQ ID NO:10); 325 bp),
HNF3β. (fw: 5′-acctgagtccgagtctgacc-3′ (SEQ ID NO:11);
                                rv: 5′-ggcaccttgagaaagcagtc-3′ (SEQ ID NO:12); 345 bp),
HNF1α. (fw: tcacagacaccaacctcagc-3′ (SEQ ID NO:13);
                                rv: 5′-gaggacactgtgggactggt-3′ (SEQ ID NO:14); 202 bp),
HNF1β. (fw: 5′-cctaggctccaacttggtca-3′ (SEQ ID NO:15);
                                rv: 5′-tgtagcgcactcctgacatc-3′ (SEQ ID NO:16); 203 bp),
PAX6 (fw: 5′-aagagtggcgactccagaagttg-3′ (SEQ ID NO:17);
                                rv: 5′-accacacctgtatccttgcttcagg-3′ (SEQ ID NO:18); 545 p),
CD45 (C57B1/6) (fw: 5′-aactggaacactgcctgctt-3′ (SEQ ID NO:19);
                                rv: 5′-gaccacctcactgtcacgttt-3′ (SEQ ID NO:20); 198 bp),
cyclophilin (fw: 5′-cagacgccactgtcgcttt-3′ (SEQ ID NO:21);
                                rv: 5′-tgtctttggaactttgtctgcaa-3′ (SEQ ID NO:22); 132 bp)
```

Functional Characterization of Islet Cells:

I. Insulin Secretion

After FACS or manual isolation of fluorescent cells with a fluorescence microscope, islet cells (1000 cells per microtiter well) were cultured for 24 hours before further analysis. Cells were then switched to medium containing either 5.5 mM or 11.1 mM glucose for additional 10 hours. Some cells were also stimulated with the glucagon-like peptide-1 analog exendin-4 (Sigma-Aldrich, St. Louis, Mo.) (10 nM) for the last 4 hours of incubation in 11.1 mM glucose. As control cells, isolated islet cells from INS2*EGFP were taken for parallel experiments. Supernatant of islet cells was harvested, gently spun and analyzed for insulin by ELISA (Ultra Sensitive Insulin ELISA Kit, Crystal Chem, Chicago, Ill.).

II. Measurements of Intracellular Calcium ([Ca²+]ᵢ)

Single cell suspensions of islet cells were plated onto glass cover slips (25CIR-1; Fisher Scientific, St. Louis, Mo.) coated with 1 mg/ml concanavalin A (type V; Sigma-Aldrich Co., St. Louis, Mo.). Cell cultures were equilibrated in culture for 24 hours prior to experiments. Expression of EGFP was detected through use of an inverted fluorescence microscope (Eclipse TE300, Nikon Instruments, Melville, N.Y.). A liquid light guide directed unfiltered excitation light to an EGFP filter set mounted in a filter cube (HQ filter set 41028, Chroma Tech. Corp., Brattleboro, Vt.). Once an EGFP-positive cell was identified, the filter cube was switched manually to a second cube containing components of the fura-2 filter set. Excitation light provided by the xenon arc lamp was reflected by a rotating chopper mirror through 340/20BP and 380/20BP excitation filters (Chroma) mounted in a motorized filter wheel located at the light source. The filtered light was then directed to the fura-2 filter set by way of the liquid light guide. The filter cube used for measurements of fura-2 contained a 400DCLP dichroic beam splitter and a 510/80 emission filter (Chroma). Fura-2 was used to detect fluctuations in [Ca²⁺]ᵢ (Kang et al., 2001). The fura-2 loading solution consisted of a standard extracellular saline (SES) containing NaCl 138 mM, KCl 5.6 mM, CaCl2 2.6 mM, MgCl2 1.2 mM HEPES 10 mM, and D-glucose 5.6 mM. The pH was adjusted to 7.35 with NaOH and the osmolarity was adjusted to 295 mOsm using H₂O. The SES was supplemented with 1 μM fura-2 acetoxymethyl ester (fura-2, AM; Molecular Probes Inc., Eugene, Oreg.), 2% FBS, and 0.02% Pluronic F-127 (w/v; Molecular Probes Inc.). Cells were exposed to the fura-2 AM-containing solution for 20-30 min at 22° C. The loading solution was then removed, and cells were equilibrated in fresh SES for 10 min at 22° C. Under these conditions, approximately 9% of fura-2 is compartmentalized, the remaining 91% being restricted to the cytosol (Leech et al., 1994). Experiments were performed at 32° C. using a temperature-controlled stage (Medical Systems Corp. Greenvale, N.Y.), a superfusion system, and a 100×UVF oil immersion objective (Nikon, Melleville, N.Y.). Dual excitation wavelength microfluorimetry was performed ratio-metrically at 0.5 s intervals using a digital video imaging system outfitted with an intensified CCD camera (IonOptix Corp., Milton, Mass.). The average of 5 video frames of imaging data was used to calculate numerator and denominator values for determination of 340/380 ratio values. [Ca²⁺]ᵢ was calculated according to the method of Grynkiewisz (Grynkiewicz et al., 1985):

[Ca²⁺]ᵢ=K_{d}*β*[(R−Rₘᵢₙ)/Rₘₐₓ−R)]

Values of Rₘᵢₙ and Rₘₐₓ were 0.20 and 7.70 for the experiments described. Spontaneous fluctuations of [Ca²⁺]ᵢ were measured under conditions in which the SES contained either 11.1 mM D-glucose or no added glucose. Fluctuations of [Ca2+]i were also measured when cells were stimulated by local (“puffer”) application of extracellular KCl (56 mM for 30 s) or glyburide (200 nM for 30-90 s) (Holz et al., 1993).

### Example 1

### Detection of an Insulin⁺ Phenotype in Transplanted Adult Bone Marrow Derived Stem Cells

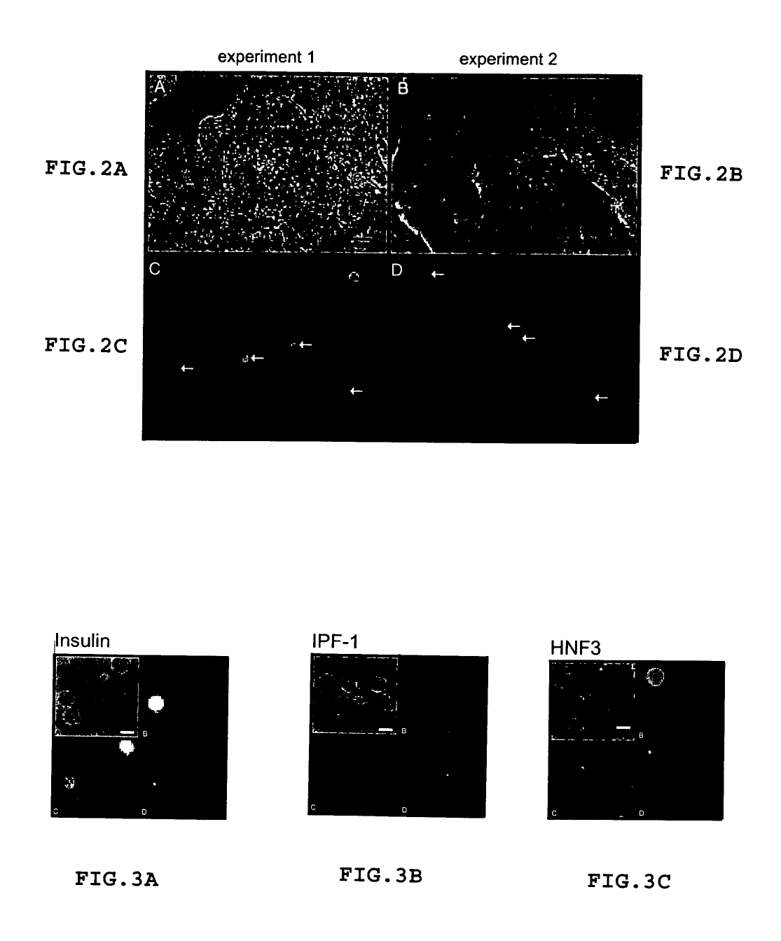

A total of 58 animals were irradiated and injected with bone marrow cells. Mortality of irradiated and bone marrow transplanted animals was approximately 50%. Results of twelve animals used in experiment 1 and thirteen animals used in experiment 2 (FIGS. 1A and 1B) are reported. Engraftment was found to be 70-90% when peripheral blood nucleated cells were examined for the presence of a Y chromosome. When wild-type female recipient animals were transplanted with bone marrow cells from male INS2*EGFP mice, EGFP-positive cells were detected within the islets of recipient mice (FIGS. 2A-2D) at 4-6 weeks after transplantation. On frozen section (FIGS. 2A-2D) as well as in single cell preparation (FIG. 3A), these EGFP-positive cells express insulin, as determined by immunohistochemistry. Furthermore, on FISH analysis, these cells revealed a Y chromosome within their nuclei, indicating that these cells were derived from the bone marrow of the male donor mouse (FIGS. 2A-2D and 3A-3C).

### Example 2

### Detection of Beta Cell Transcription Factors and a GLUT2⁺ Phenotype in Transplanted Adult Bone Marrow Derived Stem Cells

Bone marrow derived from islets expressed not only for insulin, but also transcription factors specific for beta cells (IPF-1; FIG. 3B) and endoderm (HNF3β; FIG. 3C). In contrast, when ROSA-stoplox-EGFP female mice received bone marrow transplantation from INS2-CRE male donor mice (experiment 2), none of the cells within islets or in the rest of the pancreas were fluorescent (FIGS. 2B and 2D). Nevertheless, Y chromosome positive cells were detectable within the islets, indicating that in experiment 2 engraftment of bone marrow derived cells does take place in the islet and that no recombinationevents at the LoxP sites of recipient mice are detectable (FIGS. 3A-3C).

This result indicates that cell fusion is unlikely to underlie differentiation of bone marrow derived cells into a beta cell phenotype. Fluorescence activated cell analysis (10⁴ cells counted per islet cell preparation) (FIGS. 4A-4C) or manual counting under a microscope preparation of dispersed islet cells showed that 4-6 weeks after bone marrow transplantation, the proportion of EGFP expressing cells within the islet ranged from 1.7-3% of all cells within an islet (FIG. 4C, panel C) as compared to 80-85% in islets from control INS2*EGFP mice (FIG. 4C, panel B). Neither PBNC (FIG. 4A) nor bone marrow (FIG. 4B) from donor nor recipient mice revealed detectable EGFP in any of the experiments.

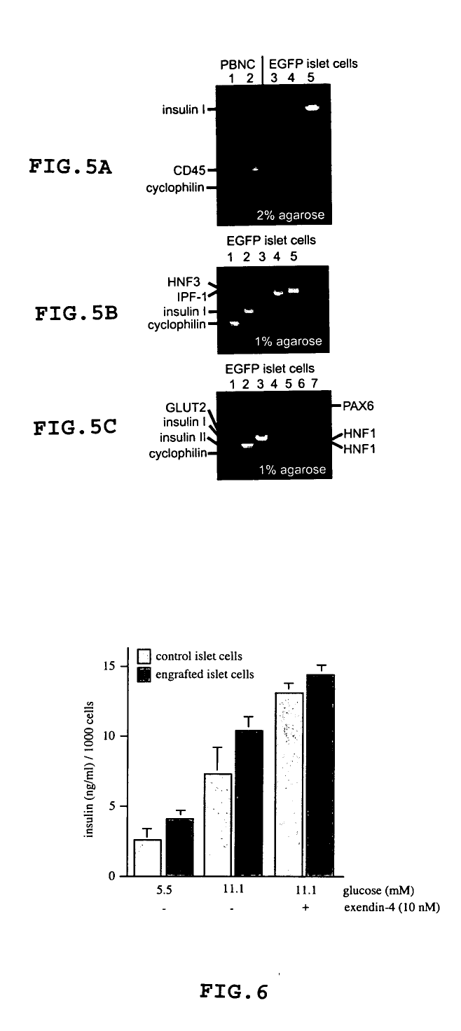

After sorting and culture for 24 hours, EGFP-positive cells from experiment 1 were further characterized by RT-PCR to express insulin I, insulin II, GLUT2, IPF-1, HNFlα, HNFlβ, HNF3β, PAX6, and lack the common hematopoietic/leucocyte marker CD45 (FIGS. 5A-5C). This suggests that the bone marrow derived cells that had engrafted the islets and expressed EGFP do not express a non-beta-cell marker present in circulating nucleated cells.

### Example 3

### Responses to Glucose

FAC-sorted cells responded to glucose as well as the glucagon-like peptide-1 synthetic analog exendin-4 with an insulin secretory response similarly to control mouse islet cells that were analyzed in parallel (FIG. 6). These results indicate that the cells studied have the machinery to sense glucose and the incretin hormone glucagon-like peptide-1 similarly to WT mouse beta cells.

### Example 4

### Calcium Signaling

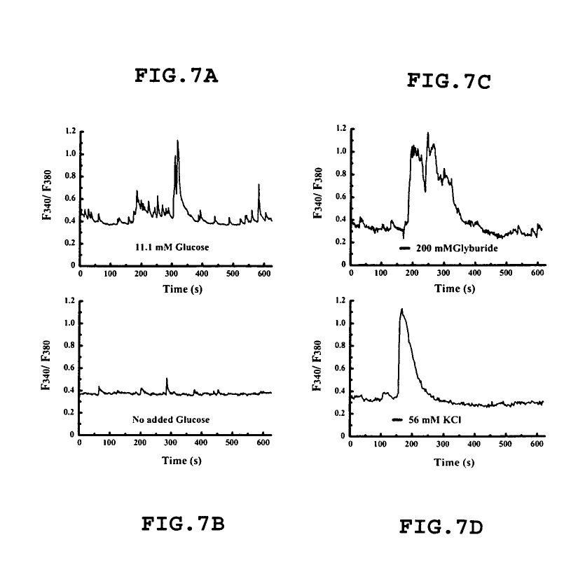

Studies examining calcium signaling in the EGFP-expressing cells were additionally performed. Spontaneous oscillations in [Ca²⁺]ᵢ were detected when single isolated cells were incubated in 11.1 mM glucose (FIG. 7A). This activity was clearly reduced when no glucose was added to the saline in which the cells were bathed. Application of a depolarizing concentration (56 mM) of extracellular KCl produced an increase in [Ca²⁺]ᵢ (FIG. 7C), suggesting that these cells possess voltage-dependent Ca²⁺ channels (VDCCs). Furthermore, application of the sulfonylurea glyburide, an inhibitor of ATP-sensitive K⁺ channels (K-ATP), produced an increase in [Ca²⁺]ᵢ (FIG. 7D). This behavior of intracellular calcium dynamics is qualitatively consistent with a pancreatic beta cell phenotype of the EGFP expressing cells and indicates that these cells possess VDCCs and K-ATP.

### Example 5

### Adminstration of Granulocyte-Macrophage Colony-Stimulating Factor (GM-CSF)

Peptides, such as granulocyte-macrophage colony-stimulating factor (GM-CSF) appear to be effective in stimulating homing of bone marrow derived cells to the pancreas. Female wild-type mice are splenectomized 2 weeks before bone marrow transplantation with adult male INS2*EGFP BM. Splenectomy is a simple, well-tolerated procedure, which preserves the pancreas in the animal. Recipient mice are treated with recombinant human GM-CSF (50ug/kg/day) for two weeks starting one day after bone marrow transplantation. Four to six weeks after bone marrow transplantation, the mice are sacrificed and relative amounts of fluorescent cells in their islets are determined with FACS analysis. Initial data indicates that administration of GM-CSF increases the proportion of BM derived fluorescent cells from 3 to 10%.

### Example 6

### Transplantation of Bone Marrow in Diabetic Mouse Model

Bone marrow from male INS2*EGFP mice was transplanted into lethally irradiated female Akita mice. Akita mice harbor a point mutation in the insulin2 gene. The protein product of this mutated insulin gene does not fold properly in the insulin producing beta-cells. The misfolded insulin2 protein causes a misfolded endoplasmatic reticulum stress in the beta-cells, which then exhibit dysfunction and apoptosis (programmed cell death). Thus, kita mice have a beta-cell autonomous and specific appoptosis and slow development of pancreatic diabetes.

Table 1 below presents fasting glucose levels (in mg/dl) in control Akita mice and in Akita mice that have received a bone marrow transplantation from a donor wild-type mouse (without a point mutation in the insulin gene).

TABLE 1

| | | Transplant recipient |
|---|---|---|
| Week after BM | Non transplanted | Akita mice |
| Transplantation | Akita mice (n = 3) | (n = 7) |
| 0 | 198 ± 12 | 204 ± 17 |
| 1 | 218 ± 2 | 193 ± 17.1 |
| 2 | 225 ± 6.7 | 191 ± 11.9 |
| 3 | 220 ± 6.2 | 184 ± 5.3 |

The pancreas from one transplanted Akita mouse was harvested after three weeks after bone marrow transplantation. FACS analysis of the isolated islet cells showed 1.3% of islet cells to be positive for green fluorescent protein. These results demonstrate that bone marrow derived cells can engraft into islets of diabetic mice, can differentiate into glucose producing beta-cells, and can ameliorate glucose metabolism and the diabetic phenotype.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Abraham E J, Leech C A, Lin J C, H. Z, Habener J F. Insulinotropic hormone glucagon-like-1 differentiation of human pancreatic islet-derived progenitor cells into insulin-producing cells. Endocrinology 2002;143(2):3152-3161.

Allen M D, Weyhrich J, Gaur L, Akimoto H, Hall J, Dalesandro J, Sai S, Thomas R, Nelson K A, Andrews R G Prolonged allogeneic and xenogeneic microchimerism in unmatched primates without immunosuppression by intrathymic implantation of CD34+ donor marrow cells Cell Immunol 1997 Nov 1;181(2):127-38

Asahara T, Masuda H, Takahashi T, Kalka C, Pastore C, Silver M, Kearne M, Magner M, Isner J M. Bone marrow origin of endothelial progenitor cells responsible for postnatal vasculogenesis in physiological and pathological neovascularization. Circ Res 1999;85(3):221-8.

Bonner-Weir S, Taneja M, Weir G C, Tatarkiewicz K, Song K H, Sharma A, O'Neil J J. In vitro cultivation of human islets from expanded ductal tissue. Proc Natl Acad Sci USA 2000;97(14):7999-8004.

Brazelton T R, Rossi F M, Keshet G I, Blau H M. From marrow to brain: expression of neuronal phenotypes in adult mice. Science 2000;290(5497):1775-9.

Czyz J, Wobus A. Embryonic stem cell differentiation: the role of extracellular factors. Differentiation 2001;68(4-5):167-74.

Dabeva M F, Hwang S G, Vasa S R et al. Differentiation of pancreatic progenitor cells into hepatocytes following transplantation into rat liver. Proc. Natl. acad Sci USA 1997;(94):7356-7361.

Fernandes A, King L C, Guz Y, Stein R, Wright C V, Teitelman G. Differentiation of new insulin-producing cells is induced by injury in adult pancreatic islets. Endocrinology 1997;138(4):1750-62.

Ferrari G, Cusella-De Angelis G, Coletta M, Paolucci E, Stornaiuolo A, Cossu G, Mavilio F. Muscle regeneration by bone marrow-derived myogenic progenitors. Science 1998;279(5356):1528-30.

Flier S N, Kulkarni R N, Kahn C R. Evidence for a circulating islet cell growth factor in insulin-resistant states. Proc Natl Acad Sci USA 2001;98(13):7475-80.

Gannon M, Shiota C, Postic C, Wright C V, Magnuson M. Analysis of the Cremediated recombination driven by rat insulin promoter in embryonic and adult mouse pancreas. Genesis 2000;26(2):139-42.

Grynkiewicz G, Poenie M, Tsien R Y. A new generation of Ca2+ indicators with greatly improved fluorescence properties. J Biol Chem 1985;260(6):3440-50.

Gu G, Dubauskaite J, Melton D A. Direct evidence for the pancreatic lineage: NGN3+ cells are islet progenitors and are distinct from duct progenitors. Development 2002;129(10):2447-57.

Gussoni E, Soneoka Y, Strickland C D, Buzney E A, Khan M K, Flint A F, Kunkel L M, Mulligan R C. Dystrophin expression in the mdx mouse restored by stem cell transplantation. Nature 1999;401(6751):390-4.

Guz Y, Nasir I, Teitelman G. Regeneration of pancreatic beta cells from intra-islet precursor cells in an experimental model of diabetes. Endocrinology 2001;142(11):4956-68.

Halvorsen T, Levine F. Diabetes mellitus-cell transplantation and gene therapy approaches. Curr Mol Med 2001;1(2):273-86.

Holz G G, Kuhtreiber W M, Habener J F. Pancreatic beta-cells are rendered glucose-competent by the insulinotropic hormone glucagon-like peptide-(7-37). Nature 1993;361(6410):362-5.

Hussain M A, Habener J F. Glucagon-like peptide 1 increases glucose-dependent activity of the homeoprotein IDX-1 transactivating domain in pancreatic betacells. Biochem Biophys Res Commun 2000;274(3):616-9.

Jackson K A, Majka S M, Wang H, Pocius J, Hartley C J, Majesky M W, Entman M L, Michael L H, Hirschi K K, Goodell M A. Regeneration of ischemic cardiac muscle and vascular endothelium by adult stem cells. J Clin Invest 2001;107(11):1395-402.

Jackson K A, Majka S M, Wang H, Pocius J, Hartley C J, Majesky M W, Entman M L, Michael L H, Hirschi K K, Goodell M A. Regeneration of ischemic cardiac muscle and vascular endothelium by adult stem cells. J Clin Invest 2001 June;107(11):1395-402.

Jiang Y, Jahagirdar B N, Reinhardt R L, Schwartz R E, Keene C D, Ortiz-Gonzalez X R, Reyes M, Lenvik T, Lund T, Blackstad M and others. Pluripotency of mesenchymal stem cells derived from adult marrow. Nature 2002;418(6893):41-9.

Kang G, Chepurny O G, Holz G G. cAMP-regulated guanine nucleotide exchange factor II (Epac2) mediates Ca2+-induced Ca2+ release in INS-1 pancreatic betacells. J Physiol 2001;536(Pt 2):375-85.

Korbling M, Katz R L, Khanna A, Ruifrok A C, Rondon G, Albitar M, Champlin R E, Estrov Z. Hepatocytes and epithelial cells of donor origin in recipients of peripheral-blood stem cells. N Engl J Med 2002;346(10):738-46.

Krause DS Regulation of hematopoietic stem cell fate. Oncogene (2002) 21, 3262-3269

Krause D S, Theise N D, Collector M I, Henegariu O, Hwang S, Gardner R, Neutzel S, Sharkis S J. Multi-organ, multi-lineage engraftment by a single bone marrow-derived stem cell. Cell 2001;105(3):369-77.

Lagasse E, Connors H, Al-Dhalimy M, Reitsma M, Dohse M, Osborne L, Wang X, Finegold M, Weissman I L, Grompe M. Purified hematopoietic stem cells can differentiate into hepatocytes in vivo. Nat Med 2000;6(11):1229-34.

Leech C A, Holz G G, Habener J F. Voltage-independent calcium channels mediate slow oscillations of cytosolic calcium that are glucose dependent in pancreatic beta-cells.

Endocrinology 1994;135(1):365-72. Lein B. Baboon bone marrow transplant suggests new directions for AIDS research. PI Perspect 1996 April;(no 18):15-7

Lin Y, Weisdorf D J, Solovey A, Hebbel R P. Origins of circulating endothelial cells and endothelial outgrowth from blood. J Clin Invest 2000;105(1):71-7.

Lipsett M, Finegood D T. beta-cell neogenesis during prolonged hyperglycemia in rats. Diabetes 2002;51(6):1834-41.

Lumelsky N, Blondel 0, Laeng P, Velasco I, Ravin R, McKay R. Differentiation of embryonic stem cells to insulin-secreting structures similar to pancreatic islets. Science 2001;292(5520):1389-94.

Mao X, Fujiwara Y, Chapdelaine A, Yang H, Orkin S H. Activation of EGFP expression by Cre-mediated excision in a new ROSA26 reporter mouse strain. Blood 2001;97(1):324-6.

Mezey E, Chandross K J, Harta G, Maki R A, McKercher S R. Turning blood into brain: cells bearing neuronal antigens generated in vivo from bone marrow. Science 2000;290(5497):1779-82.

Mohiuddin M M, Qin Y, Qian X, Meng Y, DiSesa V J Long-term survival of cardiac xenografts in fully xenogeneic (mouse-->rat) bone marrow chimeras. Ann Thorac Surg 2001 September;72(3):740-5

Orlic D, Kajstura J, Chimenti S, Jakoniuk I, Anderson S M, Li B, Pickel J, McKay R, Nadal-Ginard B, Bodine D M and others. Bone marrow cells regenerate infarcted myocardium. Nature 2001;410(6829):701-5.

Petersen B E, Bowen W C, Patrene K D, Mars W M, Sullivan A K, Murase N, Boggs S S, Greenberger J S, Goff J P. Bone marrow as a potential source of hepatic oval cells. Science 1999;284(5417):1168-70.

Rafaeloff R, Barlow S W, Rosenberg L, Vinik A I. Expression of Reg gene in the Syrian golden hamster pancreatic islet regeneration model. Diabetologia 1995;38(8):906-13.

Ramiya V K, Maraist M, Arfors K E, Schatz D A, Peck A B, Cornelius J G. Reversal of insulin-dependent diabetes using islets generated in vitro from pancreatic stem cells. Nat Med 2000;6(3):278-82.

Rao M S, Reddy J K. Hepatic transdifferentiation in the pancreas. Semin. Cell. Boil. 1995;(6):151-156.

Reddy J K, Rao M S, Qureshi S A, Reddy M K, Scarpelli D G, Lalwani N D. Induction and origin of hepatocytes in rat pancreas. J. Cell Biol. 1984;(28):2082-2090.

Reyes M, Dudek A, Jahagirdar B, Koodie L, Marker P H, Verfaillie C M. Origin of endothelial progenitors in human postnatal bone marrow. J Clin Invest 2002;109(3):337-46.

Reyes M, Lund T, Lenvik T, Aguiar D, Koodie L, Verfaillie C M. Purification and ex vivo expansion of postnatal human marrow mesodermal progenitor cells. Blood 2001;98(9):2615-25.

Rosenberg L, Vinik A I, Pittenger G L, Rafaeloff R, Duguid W P. Islet-cell regeneration in the diabetic hamster pancreas with restoration of normoglycaemia can be induced by a local growth factor(s). Diabetologia 1996;39(3):256-62.

Sauer B, Henderson N. Site-specific DNA recombination in mammalian cells by the Cre recombinase of bacteriophage P1. Proc Natl Acad Sci USA 1988;85(14):5166-70.

Schwartz R E, Reyes M, Koodie L, Jiang Y, Blackstad M, Lund T, Lenvik T, Johnson S, Hu W S, Verfaillie C M. Multipotent adult progenitor cells from bone marrow differentiate into functional hepatocyte-like cells. J Clin Invest 2002;109(10):1291-302.

Shapiro A M, Lakey J R, Ryan E A, Korbutt G S, Toth E, Warnock G L, Kneteman N M, Rajotte R V. Islet transplantation in seven patients with type 1 diabetes mellitus using a glucocorticoid-free immunosuppressive regimen. N Engl J Med 2000;343(4):230-8.

Soria B, Roche E, Berna G, Leon-Quinto T, Reig J A, Martin F. Insulin-secreting cells derived from embryonic stem cells normalize glycemia in streptozotocin induced diabetic mice. Diabetes 2000;49(2):157-62.

Soriano P. Generalized lacZ expression with the ROSA26 Cre reporter strain. Nat Genet 1999;21(1):70-1.

Stoffers D A, Kieffer T J, Hussain M A, Drucker D J, Bonner-Weir S, Habener J F, Egan J M. Insulinotropic glucagon-like peptide 1 agonists stimulate expression of homeodomain protein IDX-1 and increase islet size in mouse pancreas. Diabetes 2000;49(5):741-8.

Teitelman G. Induction of beta-cell neogenesis by islet injury. Diabetes Metab Rev 1996;12(2):91-102.

Terada N, Hamazaki T, Oka M, Hoki M, Mastalerz D M, Nakano Y, Meyer E M, Morel L, Petersen B E, Scott E W. Bone marrow cells adopt the phenotype of other cells by spontaneous cell fusion. Nature 2002;416(6880):542-5.

Theise N D, Badve S, Saxena R, Henegariu O, Sell S, Crawford J M, Krause D S. Derivation of hepatocytes from bone marrow cells in mice after radiation-induced myeloablation. Hepatology 2000;31(1):235-40.

Theise N D, Krause D S. Toward a new paradigm of cell plasticity. Leukemia 2002;16(4):542-8.

Theise N D, Nimmakayalu M, Gardner R, Illei P B, Morgan G, Teperman L, Henegariu O, Krause D S. Liver from bone marrow in humans. Hepatology 2000;32(1):11-6.

Yang L, Li S, Hatch H, Ahrens K, Cornelius J G, Petersen B E, Peck A B. In vitro trans-differentiation of adult hepatic stem cells into pancreatic endocrine hormone-producing cells. Proc Natl Acad Sci USA 2002;99(12):8078-83.

Ying Q L, Nichols J, Evans E P, Smith A G. Changing potency by spontaneous fusion. Nature 2002;416(6880):545-8.

Zulewski H, Abraham E J, Gerlach M J, Daniel P B, Moritz W, Muller B, Vallejo M, Thomas M K, Habener J F. Multipotential nestin-positive stem cells isolated from adult pancreatic islets differentiate ex vivo into pancreatic endocrine, exocrine, and hepatic phenotypes. Diabetes 2001;50(3):521-33.

What is claimed is:

1. An isolated subpopulation of bone marrow cells which are CD45⁻, Lin⁻, and Sca⁺ and which are capable of differentiating into insulin-producing pancreatic islet cells.

2. The isolated subpopulation of bone marrow cells which is human.

3. A cell isolated from the subpopulation of bone marrow cells of claim 1 which is capable of differentiating into an insulin-producing pancreatic islet cell.

4. A composition comprising the isolated subpopulation of bone marrow cells of claim 1 and a pharmaceutically-acceptable carrier, excipient, diluent or auxiliary agent.

5. A method for treating a diabetic condition in a mammal, comprising administering to the mammal a therapeutically effective amount of bone marrow, or an effective subpopulation thereof in accordance with claim 1.

6. A method in accordance with claim 5, wherein the mammal is a human.

7. A method in accordance with claim 5, wherein the diabetic condition is type I diabetes.

8. A method in accordance with claim 5, wherein the diabetic condition is type II diabetes.

9. A method in accordance with claim 5, wherein the diabetic condition is a form of secondary diabetes selected from the group consisting of pancreatic diabetes, extrapancreatic/endocrine diabetes, drug-induced diabetes, lipoatropic diabetes, myotonic dystrophy-associated diabetes and diabetes induced by disturbance of insulin receptors.

10. A method in accordance with claim 5, wherein the bone marrow is autologous.

11. A method in accordance with claim 5, wherein the bone marrow is non-autologous.

12. A method in accordance with claim 11, wherein the non-autologous bone marrow is syngeneic or allogeneic bone marrow.

13. A method in accordance with claim 5, wherein said effective subpopulation comprises a cellular composition consisting of greater than 20% bone marrow cells which are depleted of hematopoietic cells and matured leukocytes, wherein said bone marrow cells have a phenotype of CD45⁻, Lin⁻, and Sca⁺, as determined by RT-PCR, antibody staining and flow cytometry.

14. A method in accordance with claim 5, wherein said bone marrow, or effective subpopulation thereof is administered in combination with purified recombinant granulocyte colony-stimulating factor (G-CSF) or granulocyte macrophage-colony stimulating factor (GM-CSF) in an amount effective to stimulate mobilization of cells from bone marrow and differentiation into pancreatic islet cells.

15. A method for stimulating the mobilization and differentiation of bone marrow derived cells into pancreatic islet cells, comprising treating bone marrow-derived cells with an effective stimulating amount of G-CSF and/or GM-CSF.


SEQUENCE LISTING (22 sequences)

SEQ ID NO: 1 - DNA, 24 bp, Artificial
  synthetic
gcgagggcgatgccacctacggca

SEQ ID NO: 2 - DNA, 24 bp, Artificial
  synthetic
gggtgttctgctggtagtggtcgg

SEQ ID NO: 3 - DNA, 24 bp, Artificial
  synthetic
taaagatatctcacgtactgacgg

SEQ ID NO: 4 - DNA, 24 bp, Artificial
  synthetic
tctctgaccagagtcatccttagc

SEQ ID NO: 5 - DNA, 22 bp, Artificial
  synthetic
tagtgaccagctataatcagag

SEQ ID NO: 6 - DNA, 19 bp, Artificial
  synthetic
acgccaggtctgaaggtcc

SEQ ID NO: 7 - DNA, 19 bp, Artificial
  synthetic
ccctgctggccctgctctt

SEQ ID NO: 8 - DNA, 20 bp, Artificial
  synthetic
aggtctgaaggtcacctgct

SEQ ID NO: 9 - DNA, 20 bp, Artificial
  synthetic
tgtaggcagtacgggtcctc

SEQ ID NO: 10 - DNA, 20 bp, Artificial
  synthetic
ccaccccagtttacaagctc

SEQ ID NO: 11 - DNA, 20 bp, Artificial
  synthetic
acctgagtccgagtctgacc

SEQ ID NO: 12 - DNA, 20 bp, Artificial
  synthetic
ggcaccttgagaaagcagtc

SEQ ID NO: 13 - DNA, 20 bp, Artificial
  synthetic
tcacagacaccaacctcagc

SEQ ID NO: 14 - DNA, 20 bp, Artificial
  synthetic
gaggacactgtgggactggt

SEQ ID NO: 15 - DNA, 20 bp, Artificial
  synthetic
cctaggctccaacttggtca

SEQ ID NO: 16 - DNA, 20 bp, Artificial
  synthetic
tgtagcgcactcctgacatc

SEQ ID NO: 17 - DNA, 23 bp, Artificial
  synthetic
aagagtggcgactccagaagttg

SEQ ID NO: 18 - DNA, 25 bp, Artificial
  synthetic
accacacctgtatccttgcttcagg

SEQ ID NO: 19 - DNA, 20 bp, Artificial
  synthetic
aactggaacactgcctgctt

SEQ ID NO: 20 - DNA, 21 bp, Artificial
  synthetic
gaccacctcactgtcacgttt

SEQ ID NO: 21 - DNA, 19 bp, Artificial
  synthetic
cagacgccactgtcgcttt

SEQ ID NO: 22 - DNA, 23 bp, Artificial
  synthetic
tgtctttggaactttgtctgcaa